(19) United States
(12) Patent Application Publication
(10) Pub. No.: US 2010/0125340 A1
(43) Pub. Date: May 20, 2010

(54) Acetabular cup with supplemental screw fixation using conical interference fit between screw and cup

(76) Inventor: Keith J. Ure, Mt. Shasta City, CA (US)

Correspondence Address: MICHAEL J. URE, 10518 PHIL PLACE, CUPERTINO, CA, 95014, US

(21) Appl. No.: 12/271,815

(22) Filed: Nov. 14, 2008

(51) Int. Cl. A61F 2/32; A61B 19/00; A61B 17/04

(52) U.S. Cl. 623/22.36; 128/898; 606/301

(57) ABSTRACT

A fixation technique is provided whereby a solid metal acetabular cup, such as is used with hip resurfacing implants, or so called “metal on metal” bearings used with traditional total hip replacement, which do use supplemental screw fixation, can achieve immediate fixation to the acetabulum using a acetabular fixation screw, which couples to an acetabular metal cup by way of a precision taper coupling (tapered interference fit) between the fixation screw and the acetabular cup. Under normal loading, the cold weld achieved between the two surfaces is stable, and disassociation between the two metal surfaces can be achieved only with significant force.

### FIELD OF THE INVENTION

The present invention relates to joint implants, particularly hip joint implants.

### BACKGROUND OF THE INVENTION

Prior art efforts for acetabular cup fixation have included peripheral fins, dual geometry cups, spikes on the outer surface of the cup, porous outside cup surfaces which create friction between bone and socket, and a so called “press fit technique” whereby an acetabular cup is impacted into the bony acetabulum which has been reamed to a size 1-2 mm smaller than the cup. These techniques at times do not achieve adequate fixation, and cup malpositioning or “spin-out” is not uncommon. Refinements to the press fit technique include small spikes on the outer surface of the cup, which are driven into the bony acetabulum. While these measures allow for better stabilization, they give rise to difficulties with cup positioning as well as difficulties with successfully seating the acetabular component firmly against the bony acetabulum, due to displacement of the spikes. Successful contact of the cup against the acetabulum is important to achieve ingrowth of the porous surface of the cup to the bone of the acetabulum.

### OVERVIEW

A fixation technique is provided whereby a solid metal acetabular cup, such as is used with hip resurfacing implants, or so called “metal on metal” bearings used with traditional total hip replacement, which do use supplemental screw fixation, can achieve immediate fixation to the acetabulum using a acetabular fixation screw, which couples to an acetabular metal cup by way of a precision taper coupling (tapered interference fit) between the fixation screw and the acetabular cup. Under normal loading, the cold weld achieved between the two surfaces is stable, and disassociation between the two metal surfaces can be achieved only with significant force.

Additional features and benefits of the present invention will become apparent from the detailed description, figures and claims set forth below.

### BRIEF DESCRIPTION OF THE DRAWINGS

The present invention will be understood more fully from the detailed description given below and from the accompanying drawings of various embodiments of the invention, which, however, should not be taken to limit the invention to the specific embodiments, but are for explanation and understanding only.

FIG. 1 is a perspective view of an acetabular cup and an acetabular fixation screw.

FIG. 2 is a bottom perspective view of the acetabular fixation screw.

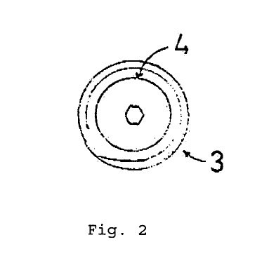

### DETAILED DESCRIPTION

Fixation arrangements for joint implants and the like are discussed.

Those of ordinary skill in the art will realize that the following detailed description of the present invention is illustrative only and is not intended to be in any way limiting. Other embodiments of the present invention will readily suggest themselves to such skilled persons having the benefit of this disclosure. It will be apparent to one skilled in the art that these specific details may not be required to practice the present invention. In the following description of the embodiments, substantially the same parts are denoted by the same reference numerals.

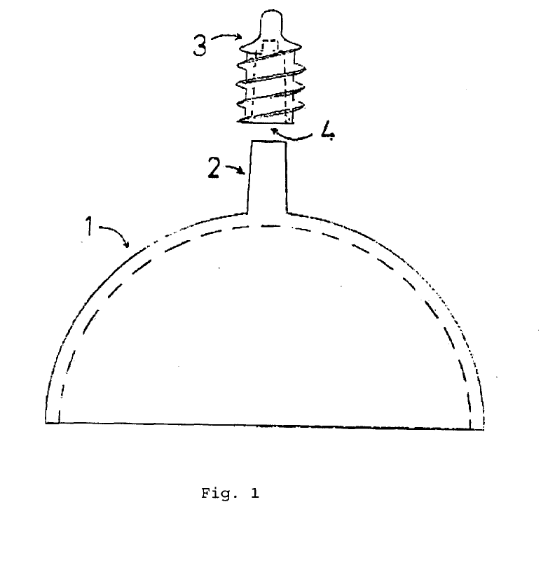

Referring now to FIG. 1, a perspective view is shown of an acetabular cup 1 and an acetabular fixation screw 3. The acetabular cup 1 is generally hemispherical and has a hollow center as indicated by the dashed line. The acetabular cup 1 is provided at a dome region thereof with a taper 2. In the illustrated embodiment, the cup portion and the taper 2 of acetabular cup 1 are integrally formed from a suitable material such as a bio-compatible metal (e.g., cobalt-chromium, titanium or other suitable metal, ceramic or other material).

The acetabular fixation screw 3 is an elongated body having, for example, threads extending therefrom. Any of various other fixation devices may used provided instead of or in addition to threads, such as “fish hooks,” a rough-textured porous coating, etc. A tapered recess 4 (indicated in hidden lines) is formed within the elongated body. At the bottom of the tapered recess 4, an engagement feature is provided for engaging a tool used to turn the elongated body. A forward end of the acetabular fixation screw 3 may have a non-threaded guide portion of a suitable length.

The taper 2 and the tapered recess 4 have surface properties that promote adhesion between their respective surfaces. For example, in instances where the acetabular cup 1 and acetabular fixation screw 3 are formed of metal, the surfaces of the taper 2 and the tapered recess 4 may be highly polished to promote adhesion (e.g., cold welding or bonding) of the surfaces to one another. The friction across the entire surface area of the interface provides a controllable amount of resistance to torque transmission, which prevents rotation of the cup or “spin out,” an unfortunate occurrence with currently available cups.

The strength of the resulting adhesion may be suitably engineered as desired. It may be desired, for example, for the adhesion to be less than maximum to allow for possible rework. In some instances, the strength of adhesion may be manually adjustable at the time of implant. For example, normally, the acetabular fixation screw would be screwed into a hole in the acetabulum so as to be even with the surface of the acetabulum, which will typically have been reamed to receive the acetabular cup. By screwing the acetabular screw into the acetabulum slightly further (e.g., a fraction of a millimeter further), the adhesion strength may be downwardly adjusted.

While particular embodiments of the present invention have been shown and described, it will be obvious to those skilled in the art that, based upon the teachings herein, changes and modifications may be made without departing from this invention and its broader aspects. Therefore, the appended claims are intended to encompass within their scope all such changes and modifications as are within the true spirit and scope of this invention.

What is claimed is:

1. A method of securing a solid metal acetabular cup, of metal on metal bearing technology, comprising achieving fixation by means of a taper coupling between said cup and a supplemental screw placed within the bone of the acetabulum with a corresponding tapered hole in its center.

2. The method of claim 1, comprising inserting the supplemental screw with a tapered hole into a dome portion of a patient's acetabulum.

3. The method of claim 2, comprising achieving an interference fit between an acetabular cup with a tapered post located at a dome region of the acetabular cup and a coupling screw (3) with hole of a corresponding taper, achieving immediate frictional fixation;
wherein friction across the entire surface area of the interface provides a controllable amount of resistance to torque transmission, which prevents rotation of the cup.

4. A method of securing an acetabular cup having a tapered projection, comprising:
anchoring within a bone structure a receiving member having a tapered recess; and
fitting the tapered projection of the acetabular cup within the tapered recess to achieve fixation.

5. An acetabular cup comprising:
a cup portion; and
a tapered projection extending from a region of the cup portion, a surface of the tapered projection having surface properties that promote adhesion between the surface of the tapered projection and a surface of a tapered recess of a receiving member.

6. The acetabular cup of claim 5, wherein the cup portion and the tapered projection are integrally formed.

7. The acetabular cup of claim 5, wherein the tapered projection is formed of metal.

8. The acetabular cup of claim 7, wherein the cup portion is formed of metal.

9. A receiving member for securing an acetabular cup, comprising:
an elongated body; and
a tapered recess formed within the elongated body, a surface of the tapered recess having surface properties that promote adhesion between the surface of the tapered recess and a surface of a tapered projection of the acetabular cup.

10. The receiving member of claim 9, comprising an engagement feature within the tapered recess for engaging a tool used to insert the elongated body into a bone structure.

11. The receiving member of claim 9, wherein the elongated body comprises a non-threaded guide portion at a leading end thereof.

12. The receiving member of claim 9, wherein the receiving member is formed of metal.

13. The acetabular cup of claim 5, wherein the tapered projection extends from a dome region of the cup portion

14. The receiving member of claim 9, wherein the elongated member has threads extending therefrom.
